(19) United States
(12) Patent Application Publication
(10) Pub. No.: US 2020/0000544 A1
(43) Pub. Date: Jan. 2, 2020

(54) DEVICE FOR REPEATABLE HEAD AND NECK TUMOR ALLOCATION DURING ONCOLOGY RADIATION THERAPY

(71) Applicants: The Trustees of Indiana University, Indianapolis, IN (US); The United State of America as Represented by the Department of Veterans Affairs, Washington, DC (US)

(72) Inventors: Jie Chen, Carmel, IN (US); Ronald Howard Shapiro, Carmel, IN (US); Bruce LuLu, Zionsville, IN (US)

(21) Appl. No.: 16/455,920

(22) Filed: Jun. 28, 2019

Related U.S. Application Data
(60) Provisional application No. 62/691,993, filed on Jun. 29, 2018.

(51) Int. Cl. A61B 90/14; A61N 5/10; A61B 6/04

(52) U.S. Cl. CPC A61B 90/14; A61N 2005/1097; A61B 6/0421; A61N 5/1049

(57) ABSTRACT

A device for accurately locate the tumor in a radiation therapy machine (RTM) based on the patient's CT image is disclosed. The device immobilizes a patient in a repeatable location when a CT scan is made and the radiation therapy is given. The device includes a base plate to support the patient, on which there are: a pair of pins to constrain the armpits of the patient, a pair of shoulder pads to constrain the shoulders of the patient, a headrest and head pads to constrain the head of the patient, markers, and a jaw restrainer secured to the head pads. Coordinate transformations will be used to locate the tumor in the RTM.

### CROSS-REFERENCE TO RELATED APPLICATIONS

This application claims priority to U.S. Provisional Patent Application No. 62/691,993, filed Jun. 29, 2018 the disclosure of which is incorporated by reference herein in its entirety.

### FIELD OF THE DISCLOSURE

The present disclosure relates to a device for patient positioning during medical treatment, or more specifically, for immobilizing the patient while receiving radiation treatment during oncology radiation therapy to enable accurate allocation of tumors.

### BACKGROUND OF THE DISCLOSURE

Radiation therapy is used to treat head and neck cancer (HNC) which accounts for 3% of all cancers in the United States. During the treatment procedure, the patient is immobilized using various devices to create reproducible patient positioning. The radiotherapy treatment process starts with a computed tomography (CT) scan in which the tumor is scanned, after which the tumor location is determined and a treatment plan is developed. The patient then receives radiotherapy treatment to eliminate the tumor. Because radiotherapy procedures such as stereotactic radiosurgery use numerous radiation beams that are precisely focused on tumors in the head and the neck, immobilization of the patient at a reproducible position is crucial for accurately location the tumors, which is critical in increasing effectiveness of the treatment and preventing the radiation beams from causing damage in a wrong part of the body.

Examples of devices used in this manner include full head-to-shoulder mask, bite blocks, occipital headrests, and head frames. However, these prior-art devices have disadvantages of their own, as explained herein. Referring to FIG. 1, a head frame 101 is used to immobilize the patient's head so at to prevent any movement during therapy. Examples of such head frames that are being used in the field include the Gill-Thomas-Cosman (GTC) frame designed for fractionated stereotactic radiotherapy, and the Brown-Roberts-Wells (BRW) frame designed for single fraction radiosurgery, among others. In FIG. 2, a bite block 102, an occipital headrest 103, and a thermoplastic mask 104 are used to prevent movement of the patient's head. In FIG. 3, another thermoplastic mask 105 designed to drape over the patient's head is used to pin down the patient's head onto a base plate 106 during radiotherapy. The difference between this mask 105 and the previous mask 104 in FIG. 2 is that the thermoplastic mask 105 lacks the opening for the patient's mouth, thereby covering the face more completely than the previous mask 104. Generally, markers are placed on the head frame 101 or masks 104 and 105 to locate and track position of the tumor relative to such markers. In the example of FIG. 2, additional markers can be placed on the bite block 102.

Numerous disadvantages exist for the abovementioned prior-art devices. For example, when using the head frame 101 as shown in FIG. 1, there can still be some relative motion between the patient's head and the head frame because the head frame has few points of contact with the patient. Also, there is considerable discomfort for the patient when wearing the head frame, and the setup is time-consuming to accurately adjust the head frame to the size and shape of the patient's head.

The prior-art setup shown in FIG. 2 uses a combination of the bite block 102, occipital headrest 103, and thermoplastic mask 104 to immobilize the patient's head. When using the bite block 102, the patient bites down on the bite block 102 so that the patient's head is immobilized with regard the bite block. However, it is very difficult for the patient to bite down on an object for a prolonged period of time due to the jaw muscle's tendency to tremble or spasm when fatigued, allowing the head to move. Also, the bite block 102 prevents the patient from breathing through the mouth, which can inhibit respiration during the treatment for patients who might have difficulty breathing through the nose due to various conditions resulting in a partially obstructed nasal canal. Furthermore, the thermoplastic mask 104 causes discomfort for patients, especially those suffering from post-traumatic stress disorder and claustrophobia, since the mask 104 covers most, if not all, of the patient's head. In some cases, excessive mental stress from such discomfort may cause the patient to panic and refuse further treatment. And even so, the setup has insufficient restraint because the patient can still manage to move his or her head when the bite block 102 and the mask 104 are applied, which causes inaccuracy in radiation dose delivery and tumor control. In addition, the setup is time-consuming and costly.

Similar problems exist for the thermoplastic mask 105, which is used as shown in FIG. 3 to immobilize the patient's head. The mask 105 can be costly to make because each patient taking radiotherapy needs to have the mask custom-made to specifically fit his or her facial profile, and therefore cannot be reused for other patients. As such, when the patient experiences weight loss during the treatment, a new mask would need to be made each time, which is not only costly but can delay the treatment if the mask is not delivered on time. The thermoplastic masks 104 and 105 also need to be replaced often because they can become brittle after extensive use.

Therefore, in view of the above, there is a need for a low-cost device with quick setup time to ensure that the tumor detected by the CT scan can be accurately located in the radiotherapy machine with minimum discomfort to the patient.

### SUMMARY OF THE DISCLOSURE

The present disclosure describes systems and devices used therein for use in positioning a patient during radiotherapy in such a way that the tumors can be precisely allocated. The fundamental requirement is to ensure the body, the neck, and the head are in repeatable positions when the patient goes through CT scan and is under treatment by a radiation therapy machine (RTM). Once the body and head are in repeatable positions, the neck will be in repeatable position. In one embodiment, the device includes a base plate, or bed, that can position a patient's body and head in repeatable locations, which ensures the tumor to have a fixed position relative to a coordinate system defined by three markers attached to the bed. The three markers are included in the patient's CT images, through which the location of the tumor can be identified and its location relative to the coordinate system defined by the three markers can be determined. Then, a treatment plan can be made based on the coordinate system defined by the markers. During the treatment stage, the three markers will be located by the RTM, an example of which is a linear accelerator (LINAC). The coordinate system defined by the three markers can be reconstructed and the location of the tumor in the system can be known. Finally, the location of the tumor can be expressed in the RTM through methods such as coordinate transformation. With accurate location of the tumor, the treatment plan can be executed effectively and the damage to the surrounding tissues will be minimized.

### BRIEF DESCRIPTION OF THE DRAWINGS

The features of this invention, and the manner of attaining them, will become more apparent and the invention itself will be better understood by reference to the following description of embodiments of the invention taken in conjunction with the accompanying drawings. In the figures, like reference numerals represent like elements, and the figures are to be understood as illustrative of the disclosure. The figures are not necessarily drawn to scale and are not intended to be limiting in any way.

FIG. 1 is a photograph showing a prior-art setup using a head frame on a patient;

FIG. 2 is a pair of photographs showing a prior-art setup using a bite block, occipital headrest, and thermoplastic mask on a patient, taken from two different angles;

FIG. 3 is a photograph showing a prior-art setup using a different thermoplastic mask attached to a base plate on a patient, taken from the side;

FIG. 4 is a top view of one embodiment of a device as described herein to immobilize a patient; and

FIG. 5 is a 3D view of a frame which supports a removable probe as used in the embodiment shown in FIG. 4.

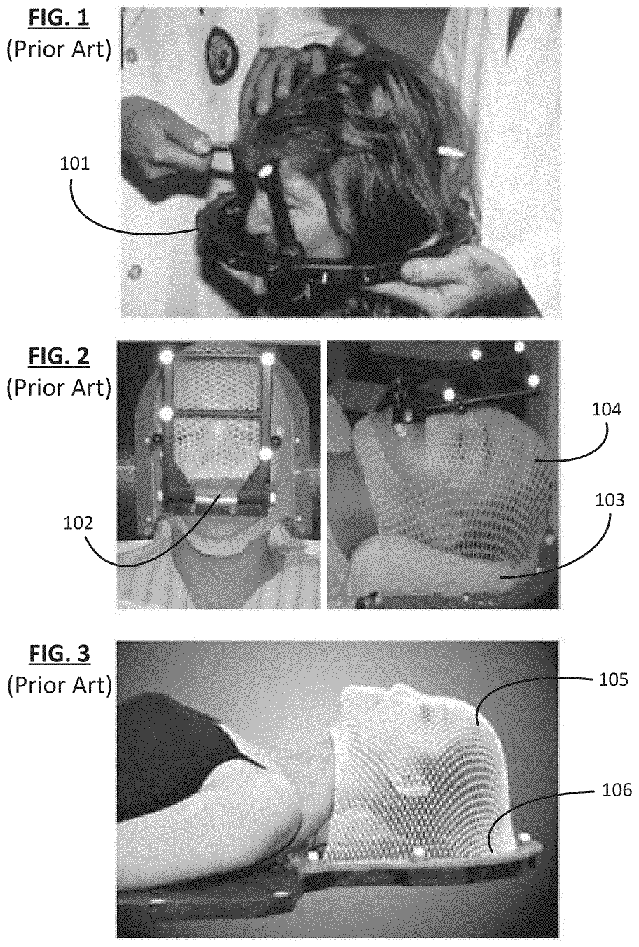

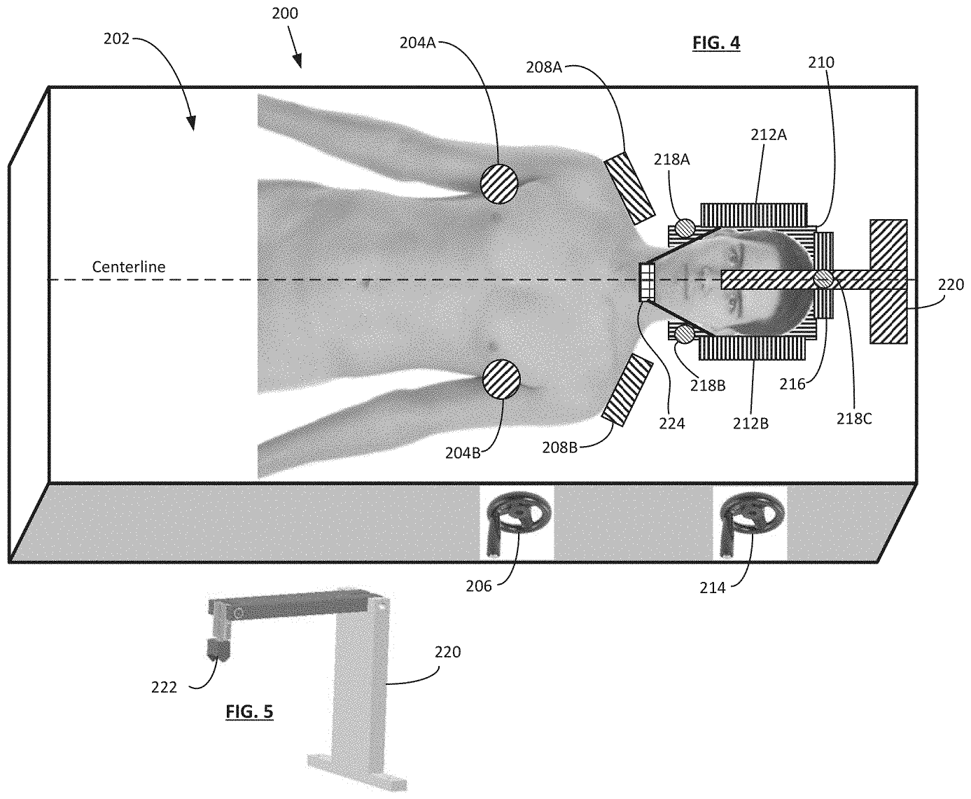

While the present disclosure is amenable to various modifications and alternative forms, specific embodiments have been shown by way of example in the drawings and are described in detail below. The intention, however, is not to limit the present disclosure to the particular embodiments described. On the contrary, the present disclosure is intended to cover all modifications, equivalents, and alternatives falling within the scope of the present disclosure as defined by the appended claims.

### DETAILED DESCRIPTION OF THE DISCLOSURE

In the following detailed description, reference is made to the accompanying drawings which form a part hereof, and in which is shown by way of illustration specific embodiments in which the present disclosure is practiced. These embodiments are described in sufficient detail to enable those skilled in the art to practice the present disclosure, and it is to be understood that other embodiments can be utilized and that structural changes can be made without departing from the scope of the present disclosure. Therefore, the following detailed description is not to be taken in a limiting sense, and the scope of the present disclosure is defined by the appended claims and their equivalents.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments. Furthermore, the described features, structures, or characteristics of the subject matter described herein may be combined in any suitable manner in one or more embodiments.

FIG. 4 illustrates one embodiment of a device 200 for immobilizing a patient. The device 200 includes a base plate 202, or a bed, on which the patient lies during radiotherapy. In one example, the base plate 202 itself can be the bed, or a table, that is slid into the CT scanner or the radiation therapy machine (RTM). One example of such RIM is a linear accelerator (LINAC). In another example, the base plate 202 is a separate component from the bed which can be attached onto the top surface of the bed, where the base plate 202 is adjustable so that it is compatible with different sizes and dimensions of the beds used in radiotherapy. The device 200 consists of mechanisms to lock the body and the head in repeatable positions. The base plate 202 has a pair of pins 204A and 204B movable toward the patient's body when the patient lies down on the base plate 202, such that each of the pins 204 can be positioned against the patient's armpits and locked in that position. The pins 204 move in a concentric motion, i.e. inward toward the center line of the base plate, and the moving and locking of the pins 204 are controlled by an indexed dial 206 located on the side of the base plate 202, in a location that does not interfere with the radiotherapy.

The base plate 202 also has a pair of movable shoulder pads 208A and 208B, which can be indexed and locked after constraining the patient's shoulders. The indexing of the shoulder pads 208 can be done, for example, using ruler grids printed on the base plate 202 that indicate the exact position on the surface of the base plate 202 onto which the shoulder pads 208 are placed, such that the exact position of the shoulder pads 208 can easily be reproduced. In one example, the base plate 202 can have a set of grooves to slide the shoulder pads 208 across, or slots to insert a part of the shoulder pads 208 into, to align with the patient's shoulders. The shoulder pads 208 can then be locked to prevent movement of the shoulders. The components 204A, 204B, 208A and 208B are used to position the body.

The base plate 202 has a headrest 210 placed at the position where the patient's head is to be laid, which constrains rotation of the back side of the head. The headrest 210 can be made of soft moldable material such as memory foam so that the headrest 210 can accommodate for the various shapes and sizes of the heads for different patients. Adjacent to the headrest 210 are two head pads 212A and 212B located to prevent movement of the head in the lateral direction, and a single head pad 216 located to prevent movement of the head in the vertical direction, with respect to the patient. On the side of the base plate 202 is another indexed dial 214 which controls movement and locking of the head pads 212. The head pads 212 move in a concentric motion, i.e. inward toward the center line of the base plate, such that they sandwich the patient's head from both sides, after which they are locked by the indexed dial 214. The other head pad 216 can be moved to align with the top of the patient's head and locked in position. Similar to the shoulder pads 208, the position of the head pad 216 can be indexed, for example, using the ruler grids printed on the base plate 202. In one example, the base plate 202 has a set of grooves which the head pad 216 can be slid across to align with the top of the patient's head, and then locked to prevent movement of the head in the vertical direction.

Also, a set of three markers 218A, 218B, and 218C are placed on the base plate 202, which acts as the identifier for the plane on which the patient lies. Using the unique-plane assumption of the point-line-plane postulate in two-dimensional Euclidean geometry (i.e. that there is only one unique plane which passes through three non-collinear points), a set of three points is therefore sufficient to define a unique plane and coordinate system, which represents the base plate 202 on which the patient lies. Based on these markers 218, the CT scanner and the RTM can pinpoint the exact relative position of the tumor with respect to the base plate 202 and the locations of the tumors.

The base plate 202 also has a frame 220 to support a removable probe 222, as shown in FIG. 5, which includes a laser pointer to help align the patient's head to the centerline of the bed. In one example, a mark may be drawn on the patient's face, such as on the forehead or on the nose, along the centerline of the face, such that aligning the mark to the light emitted from the removable probe 222 allows the patient to be positioned exactly at the centerline of the base plate 202. The removable probe 222 also can check whether the mark on the patient's face varies when restraining the patient. Finally, a jaw restrainer 224 is placed on the patient's jaw, with each end of the jaw restrainer 224 secured and locked to the closest head pad 212, thereby restraining movement of the patient's jaw. The components 210, 212A, 212B, 216, 222, and 224 are used to position the head.

In the present embodiment, the mechanical parts in the radiation area can be made of materials such as polyether ether ketone (PEEK), which is radiolucent, so that the treatment dose will be minimally affected. However, other suitable radiolucent materials can also be incorporated, as appropriate.

Firstly, the patient has a mark drawn on the face to indicate the centerline of the face. Once the patient lies down on the base plate 202, the patient is securely immobilized using the device 200 as described above, and the removable probe 222 locates the mark on the face. In restraining the patient using each of the components of the device 200, the variation of the locations of the mark on the patient's face should be checked to ensure that the patient is aligned with the centerline of the base plate 202. This variation represents the accuracy of the device 200. Then, the probe 222 is removed and the base plate 202 is slid into the CT scanner for initial detection of the tumor. The sensors inside the CT scanner scans the patient to locate the tumor with the field of view including the markers 218 placed on the base plate 202 for later reconstruction. Using the unique-plane assumption and the coordinate system as previously explained, the tumor location is determined on the coordinate system defined by the markers 218 with respect to the unique plane using a computing device coupled to the CT scanner. Then, the computer finds the markers 218 and digitally reconstructs the coordinate system. Thus, the tumor location can be identified in the coordinate system of the RTM through coordinate transformation. After careful treatment planning and virtual simulations are performed on the computer, the patient then receives radiotherapy treatment to eliminate the tumor.

Advantages of using the device 200 include ensuring that the patient's body and the head are always fully constrained in the same positions on the bed during different stages of radiotherapy, and ensuring that the relative position of the bed, as defined by the three markers located on the bed, can be located accurately with respect to the CT scanner and the RTM. Because the location of the tumor is expressed in the frame defined by the three markers, the location of tumor in the CT scanner and RTM can be identified through coordinate transformation. The device 200 also reduces the cost and setup time for radiotherapy.

The present subject matter may be embodied in other specific forms without departing from the scope of the present disclosure. The described embodiments are to be considered in all respects only as illustrative and not restrictive. Those skilled in the art will recognize that other implementations consistent with the disclosed embodiments are possible.

What is claimed is:

1. A device for immobilizing a patient in a repeatable location, comprising:
a base plate configured to support the patient;
a pair of pins configured to lock after constraining armpits of the patient;
a pair of shoulder pads configured to lock after constraining shoulders of the patient;
a headrest configured to constrain rotation of a back side of a head of the patient;
a plurality of head pads configured to lock after constraining the head;
at least three markers configured to be placed on the base plate; and
a jaw restrainer configured to be secured to the pair of head pads.

2. The device of claim 1, further comprising a frame configured to support a removable probe with a laser pointer operable to align the head to a centerline of the base plate.

3. The device of claim 1, further comprising:
a first indexed dial mechanically coupled to the pair of pins, the first indexed dial configured to control movement of the pins and lock after constraining the armpits; and
a second indexed dial mechanically coupled to the head pads, the second indexed dial configured to control movement of the head pads and lock after constraining the head.

4. A system for radiotherapy, comprising:
a scanner for scanning a body of a patient for at least one tumor;
a device for immobilizing the patient in a repeatable location a base plate configured to support the patient, the device comprising:
a pair of pins configured to lock after constraining armpits of the patient,
a pair of shoulder pads configured to lock after constraining shoulders of the patient,
a headrest configured to constrain rotation of a back side of a head of the patient,
a plurality of head pads configured to lock after constraining the head,
at least three markers configured to be placed on the base plate to be included and identifiable in a field of view of the scanner, and
a jaw restrainer configured to be secured to the pair of head pads; and
a computing device coupled to the scanner, the computing device configured to determine a location of the tumor from the body scan and record the location in a local coordinate system (LCS) defined by the markers, wherein the location of the tumor is interchangeable between a coordinate system on the scanner and the LCS using a coordinate transformation matrix.

5. The system of claim 4, further comprising:
a radiation therapy machine (RTM) configured to receive the patient; and
a computing device coupled to the RTM, the computing device configured to determine the location of the tumor from the body scan and transform the tumor location to a coordinate system on the RTM using the coordinate transformation matrix.
